(19) United States
(12) Patent Application Publication
(10) Pub. No.: US 2025/0169837 A1
(43) Pub. Date: May 29, 2025

(54) GUIDEWIRE AND SNARE APPARATUS

(71) Applicant: Bard Peripheral Vascular, Inc., Franklin Lakes, NJ (US)

(72) Inventors: Matt CASIRARO, Tempe, AZ (US); Andrzej J. CHANDUSZKO, Chandler, AZ (US)

(21) Appl. No.: 18/834,296

(22) PCT Filed: Feb. 11, 2022
(86) PCT No.: PCT/US2022/070639

§ 371 Date: Jul. 30, 2024

(51) Int. Cl. A61B 17/22; A61B 90/00

(52) U.S. Cl. CPC A61B 17/22031; A61B 90/03; A61B 2017/22035; A61B 2017/22038; A61B 2090/034

(57) ABSTRACT

A surgical guidewire and snare apparatus comprising of an elongated sheath that has a central, longitudinal bore, proximal and distal sheath ends. A head is at the sheath distal end. A wire or cable occupies the central, longitudinal bore, and the wire or cable has a proximal wire or cable and a distal wire or cable end. The wire or cable distal end has an engagement with the head. Slotted portions are on the sheath at the sheath distal end. Spaced apart sections of the sheath are next to the slotted portions. Tension is applied to the wire or cable proximal end that pulls the head proximally to enlarge the slotted portions and curve the spaced apart sheath sections next to the slotted portions.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### 1. Field of the Invention

The present invention relates to an improved guidewire apparatus that can convert to a snare if needed. More particularly, the present invention relates to a surgical guidewire and snare apparatus having a specially configured sheath with a distal end portion having slots that are spaced between sheath segments, wherein an internal wire or cable can be pulled to bend the sheath segments forming a snare. In a set of embodiments, the internal wire can also be advanced, forming an additional snare of a smaller size or distinct shape.

### 2. General Background of the Invention

Endovascular snares have traditionally been used to retrieve vena cava filters, reposition indwelling catheters, fibrin sheath stripping, assist in central venal access venipuncture and retrieve or adjust other foreign objects. Commercially available snares that exist today include the EN Snare® Endovascular Snare System by Merit Medical, ONE Snare® Endovascular Snare System by Merit Medical, and Bard® Snare.

All of these snares require a separate snare sheath where the snare is collapsed during tracking and delivery and once at the intended location the snare is advanced out of this snare sheath. This snare sheath is still introduced into a true procedural introducer sheath.

In a typical retrieval procedure, a short access sheath is used to gain an access to a vessel. A guidewire with a guiding catheter is then used to track the guidewire to the desired location. A guiding catheter may be one such as ConcierGE® Guiding Catheters by Merit Medical, available at medicalexpo.com/prod/merit-medical-systems/product-81470-520362.html. Once the guidewire is in place then a dilator would be inserted into a retrieval sheath and then both would be advanced over the guidewire to the desired location. The dilator and guidewire would then be removed, and a snare kit would be introduced via the retrieval sheath. A foreign object would be captured by pulling the snare loop into the snare catheter. The retrieval sheath would then be advanced over the snare and the foreign object or snare and object would be pulled into the retrieval sheath. The object and snare would be removed from the body via the retrieval sheath. Depending on what is captured, an additional larger diameter retrieval sheath may be utilized in conjunction with the retrieval sheath. For example, the additional sheath may be utilized in the capturing of filter hooks. A retrieval kit as is known in the art is seen by the Bard® Snare Retrieval Kit (PF10009-BAW1313100R2 Bard Snare Retrieval Kit English Only IFU, available at bd.com/assets/documents/PDH/PF10009-BAW1313100R2%20Bard%20Snare%20Retrieval%20Kit%20English%20Only%20IFU.pdf, which is hereby incorporated by reference). Parts from such a retrieval kit may be utilized with the present invention during a retrieval procedure, with the dimensions altered, as necessary.

The following possibly relevant U.S. patents are incorporated herein by reference:

| Patent or | | |
|---|---|---|
| Publication No. | Title | Issue Date |
| 5,387,219 | MEDICAL RETRIEVAL SNARE | Feb. 7, 1995 |
| | WITH COIL WRAPPED LOOP | |
| 6,299,612 | WIRE LOOP TYPE | Oct. 9, 2001 |
| | INSTRUMENT FOR | |
| | ENDOCSCOPE AND METHOD | |
| | OF PRODUCING THE SAME | |
| 6,554,842 | SMALL DIAMETER SNARE | Apr. 29, 2003 |
| 6,652,536 | SNARE WITH ANTI-SKEWING | Nov. 25, 2003 |
| 7,655,013 | SNARE RETRIEVAL DEVICE | Feb. 2, 2010 |
| 8,328,842 | FILTER ELEMENT WITH | Dec. 11, 2012 |
| | RETRACTABLE | |
| | GUIDEWIRE TIP | |
| 8,852,204 | MEDICAL SNARE DEVICE | Oct. 7, 2014 |
| 9,308,074 | FILTER RETRIEVAL DEVICE | Apr. 12, 2016 |
| 2004/0116960 | ONE PIECE LOOP AND COIL | Jun. 17, 2004 |
| 2005/0234474 | SMALL-DIAMETER SNARE | Oct. 20, 2005 |
| 2006/0100544 | STEERABLE LOOP TIP | May 11, 2006 |
| | WIRE-GUIDE | |
| 2009/0240238 | CLOT RETRIEVAL | Sep. 24, 2009 |
| | MECHANISM | |
| WO2012/031149 | MEDICAL SNARE DEVICE | Mar. 8, 2012 |

### BRIEF SUMMARY OF THE INVENTION

The present invention is a guidewire that can convert to a snare if needed. The guideline apparatus can be between 0.010 inches (0.254 mm) and 0.038 inches (0.965 mm) (e.g., 0.035 inches) in diameter. The guidewire and snare apparatus has an outer sheath with a longitudinal bore or lumen and a wire or cable that slides in the bore or lumen. The tip of the apparatus can be rounded or hemispherical (e.g., domed) as typical but can be attached to one or more stainless steel (or Nitinol or Cobalt Chromium or other metal alloys or carbon fiber) braided cables via laser, plasma or solder welding process. Alternatively, the tip may be formed via laser, plasma or solder treatment in a fixture to produce an atraumatic shape (e.g., domed or hemispherical). The tip may have a soft extension as used in regular guidewires and be either straight or curved (e.g. J-shaped). The braided wire or cable could be of 7×7 construction with an approximate diameter of 0.0106″ (0.27 mm) which results in a tensile breaking load of 13.26 lbf (59N). The wires or cables could be coated with a thin layer of gold or tungsten or other radiopaque material. The sheath could be produced so that slotted portions extend longitudinally along the distal end portion. The distal snare part can be wire or cable or different material than the proximal shaft, which can be a single wire. The outer sheath is preferably Nitinol.

The tensile breaking load of approximately 13.26 lbf (59N) would be much greater than the typical maximum acceptable pulling load 5 lbf that is seen clinically thus providing a significant factor of safety.

During clinical use (for a hypothetical foreign object removal), the inner guidewire would be used in conjunction with the outer sheath. In various embodiments, the guidewire would be introduced with the cable tip in contact with the distal sheath face and then tracked to the location for the removal. As the wire or cable is pulled relative to the sheath, snare loops are formed on the sheath (oval or round shape). The foreign object would be snared and pulled into the retrieval sheath completing the procedure.

There are multiple advantage of the present invention over prior art and commercially available snares.

The apparatus of the present invention eliminates the need of having both a snare and a snare sheath, as the uniquely configured apparatus provides a snare that is part of a sheath, rather than separate therefrom, thus reducing the profile of the device. Additionally, the apparatus can be used as a guidewire thus reducing cost and extra procedural steps (reduces complexity).

Since a guidewire is needed anyways to track, the present invention can completely eliminate the need for a separate snare product code reducing procedural costs and simplifying inventory.

In the present invention, the profile of the introducer sheath utilized is reduced since the snare sheath no longer needs to fit so there is room to go smaller in profile. With the present invention, a reduction of the introducer sheath of at least 2F is possible (could reduce further but will depend on application as foreign object removal requires some space for object to be removed in procedural sheath).

If additional procedural tasks are needed, the guidewire could be used which eliminates the need to pull an additional guidewire.

Given the lower profile of the present invention due to a smaller sheath and wire, the potential exists for this to be used to capture tibiopedal retrograde wire for CTO (chronic total occlusion) crossing. During this procedure access is gained in tibials or pedals and crossing is done retrograde while simultaneously femoral access is gained above for subsequent atherectomy or other therapy. A wire is used to cross retrograde using the tibiopedal access. Once across the CTO the proposed invention (guidewire with snare) could snare the retrograde CTO crossing wire that has come from below.

In various embodiments, a surgical guidewire and snare apparatus has an elongated sheath having a central, longitudinal bore and proximal and distal sheath ends. There is a head at the sheath distal end. A wire or cable occupies the central, longitudinal bore of the sheath, the wire or cable having a proximal wire or cable end and a distal wire or cable end. In various embodiments, the wire or cable distal end has an engagement or attachment with the head.

There are slotted portions on the sheath at the sheath distal end. Spaced apart sections of the sheath are next to the slotted portions. Applying tension to the wire or cable proximal end pulls the head proximally to enlarge the slotted portions and curve the spaced apart sheath sections next to the slotted portions.

During this procedure, a physician or technician could hold the sheath with one hand and pull the wire or cable with the other hand (e.g., using a commercially available guidewire operator such as the Merit® Torque Device, catalog number MAP500/MAP501, available at merit.com/cardiac-intervention/angiography/guide-wire-accessories/merit-torque-device/). The handle can attach to the sheath and have the cable or wire pass freely through it when unlocked and be fixed/pinched if locked (e.g., when a locking button is depressed). The handle need not be attached to the sheath, however. To activate the snare a physician would either pull on the cable with one hand while holding the handle still and then lock the cable position with the button or, as an alternative mechanism, the physician would use a thumb to e.g. pull the cable by pulling the button and then locking it by e.g. pressing it.

In various embodiments, the wire or cable distal end is attached to the sheath distal end.

In various embodiments, there are two sheath sections next to the slotted portions. In various embodiments, there are three sheath sections next to the slotted portions. In various embodiments there are four sheath sections next to the slotted portions.

In various embodiments, the sheath head has a longitudinal bore and the wire or cable extends through the sheath head bore, and wherein the wire or cable has an enlarged stop member positioned distally of the head that is too large to pass through the sheath head bore.

In various embodiments, the wire or cable extends through the head and has a curved portion (e.g., j-tip portion) on the wire or cable positioned distally of the head.

In various embodiments, the wire or cable extends through the head and has a curved portion (e.g., j-tip portion) on the wire or cable that is positioned distally of the stop member.

In various embodiments, the wire or cable has two or more separable sections at the wire or cable distal end portion. Alternatively, two wires or cables can be used.

In various embodiments, the sheath head has a longitudinal bore and the wire or cable extends through the sheath head bore, wherein the wire or cable has an enlarged stop member positioned distally of the head that is too large to pass through the sheath head bore.

In various embodiments, a surgical guidewire and snare apparatus has an elongated sheath having a central, longitudinal bore and proximal and distal sheath ends. There is a head at the sheath distal end. A wire or cable occupies the central, longitudinal bore, the wire or cable having a proximal wire or cable end and a distal wire or cable end. The wire or cable extends through the head and distally beyond the head. There are slotted portions on the sheath at the sheath distal end. There are spaced apart sections of the sheath next to the slotted portions. Applying tension to the wire or cable proximal end pulls the head proximally to enlarge the slotted portions and bend or curve the spaced apart sheath sections next to the slotted portions.

In various embodiments, a stop is positioned on the wire or cable distally of the sheath. Applying tension to the wire or cable proximal end pulls the stop against the head, moving the head proximally to enlarge the slotted portions and bend or curve the spaced apart sheath sections next to the slotted portions.

In various embodiments, there are multiple wires or cables in a sheath bore.

In various embodiments, the wire or cable bifurcates into two wire or cable segments wherein the wire or cable can be extended distally of the sheath head. In such a case, the wire can function as a snare distally of the sheath.

In various embodiments, the wire or cable trifurcates into three wire or cable segments wherein the wire or cable can be extended distally of the sheath head. In such a case, the wire can function as a snare distally of the sheath.

In various embodiments, the wire or cable splits into four wire or cable segments wherein the wire or cable can be extended distally of the sheath head. In such a case, the wire can function as a snare distally of the sheath.

In various embodiments, there are two sheath sections next to the slotted portions. In various embodiments, there are three sheath sections next to the slotted portions. In various embodiments, there are four sheath sections next to the slotted portions.

In various embodiments, the sheath sections are circumferentially spaced apart.

In various embodiments, the sheath head has a longitudinal bore and the wire or cable extends through the sheath head bore, wherein the wire or cable has an enlarged stop member positioned distally of the head that is too large to pass through the sheath head bore.

In various embodiments, a surgical guidewire and snare apparatus has an elongated sheath having a central, longitudinal bore and proximal and distal sheath ends. There is a head at the sheath distal end. A wire or cable occupies the central, longitudinal bore, the wire or cable having a proximal wire or cable end and a distal wire or cable end. The wire or cable extends through the head and distally beyond the head. There are slotted portions on the sheath at the sheath distal end. There are spaced apart sections of the sheath next to the slotted portions. Applying tension to the wire or cable proximal end pulls the head proximally to enlarge the slotted portions and bend or curve the spaced apart sheath sections next to the slotted portions. The distal wire or cable is bifurcated to form a wire or cable snare when the distal wire or cable end is advanced distally of the sheath distal end head.

In various embodiments, a stop is positioned on the wire or cable distally of the sheath head.

In various embodiments, a surgical guidewire and snare apparatus has an elongated sheath having a central, longitudinal bore and proximal and distal sheath ends. There is a head at the sheath distal end. A wire or cable occupies the central, longitudinal bore, the wire or cable having a proximal wire or cable end and a distal wire or cable end. The wire or cable extends through the head and distally beyond the head. There are slotted portions on the sheath at the sheath distal end. There are spaced apart sections of the sheath next to the slotted portions. Applying tension to the wire or cable proximal end pulls the head proximally to enlarge the slotted portions and bend or curve the spaced apart sheath sections next to the slotted portions. The distal wire or cable forms a wire or cable snare when the distal wire or cable end is advanced distally of the head.

In various embodiments, the distal wire or cable bifurcates into three wires or cables. In various embodiments, the distal wire or cable trifurcates into three wires or cables. In various embodiments, the distal wire or cable forks into four or more wires or cables.

In various embodiments, the distal wire or cable trifurcates into three wires or cables and the said three wires or cables are approximately 120 degrees apparat from each other when the wire or cable snare is formed by advancing the distal wire or cable distally of the head.

In various embodiments, the sheath head has a longitudinal bore and the distal wire or cable extends through the sheath head bore, wherein the distal wire or cable has an enlarged stop member positioned distally of the head that is too large to pass through said sheath head bore.

In various embodiments, a wire or cable extends distally of the stop member.

In various embodiments, the wire or cable extending distally of the stop member is a straight.

In various embodiments, the wire or cable extending distally of the stop member has a j-tip portion.

In various embodiments, the wire or cable extending distally of the stop member is angled.

In various embodiments, the wire or cable extending distally of the stop member has an angled portion and/or tip.

J-tips and angled guidewire extensions can help traversing various anatomy such as bifurcations (vessels that split into two) and tortuous vessels. The J-tip or angled tip helps to guide to an angled bifurcation and also through tortuous vessels (for example the wire can be rotated and the J-tip or angled tip can be used to aid the user in guiding in the intended direction). Also, a wire extension at tip can help to poke forward and assess an atherosclerotic lesion (i.e. is it soft or hard) depending on how it interacts with the lesion. An atraumatic soft tip helps to prevent potential adverse events associated with advancing endovascular devices such as vessel perforation and bleeding.

In various embodiments, the distal wire can be bent or straight or have a bent or straight portion when extending the inner wire distally of the sheath (for example, in those embodiments where the wire (guidewire) can form a separate snare). The usefulness of bent or straight wire depends on the anatomy, the object being retrieved, and how the object is positioned/angled relative to the anatomy and the snare; having different versions of the device allows a physician to select the best approach based on the aforementioned factors.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a side view of a first embodiment of the apparatus of the present invention;

FIG. 2 is a schematic, sectional view taken along lines 2-2 of FIG. 1;

FIG. 3 is a side view of a first embodiment of the apparatus of the present invention;

FIG. 4 is a perspective view of a second embodiment of the apparatus of the present invention;

FIG. 5 is a perspective view of a third embodiment of the apparatus of the present invention;

FIG. 6 is a side view of a fourth embodiment of the apparatus of the present invention;

FIG. 7 is a partial side view of a fourth embodiment of the apparatus of the present invention;

FIG. 8 is a sectional view taken along lines 8-8 of FIG. 7;

FIG. 9 is a partial side view of a fifth embodiment of the apparatus of the present invention;

FIG. 10 is a partial side view of a fifth embodiment of the apparatus of the present invention;

FIG. 11 is a side view of a fifth embodiment of the apparatus of the present invention;

FIG. 12 is a side view of a fifth embodiment of the apparatus of the present invention;

FIG. 13 is a partial side view of a sixth embodiment of the apparatus of the present invention;

FIG. 14 is a partial side view of a sixth embodiment of the apparatus of the present invention;

FIG. 15 is a partial side view of a seventh embodiment of the apparatus of the present invention;

FIG. 16 is a sectional view taken along lines 15-15 of FIG. 15;

FIG. 17 is a side view of an eighth embodiment of the apparatus of the present invention;

FIG. 18 is a top view of a eighth embodiment of the apparatus of the present invention;

FIG. 19 is a side view of a ninth embodiment of the apparatus of the present invention;

FIG. 20 is a front end view of a ninth embodiment of the apparatus of the present invention; and

FIG. 21 is a side view of a tenth embodiment of the apparatus of the present invention.

### DETAILED DESCRIPTION OF THE INVENTION

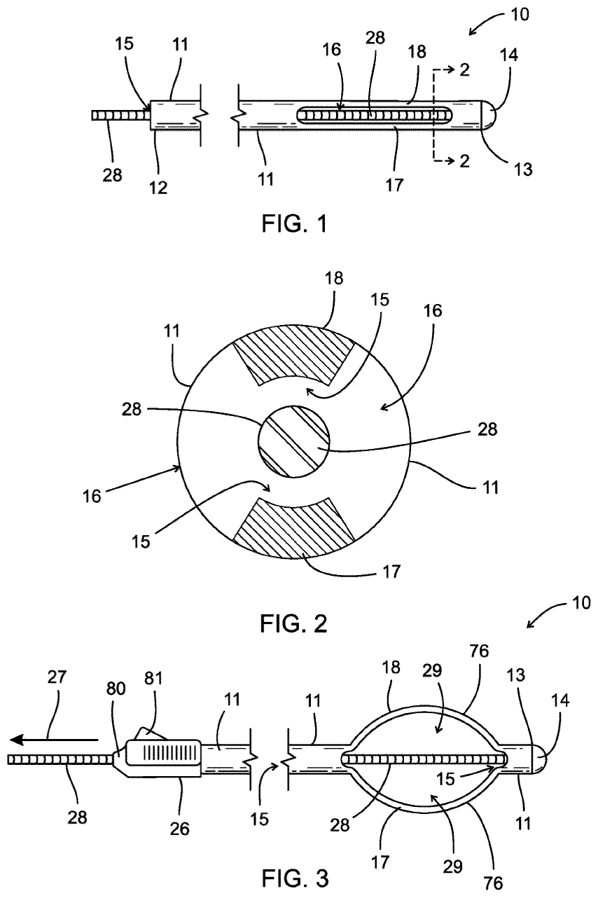

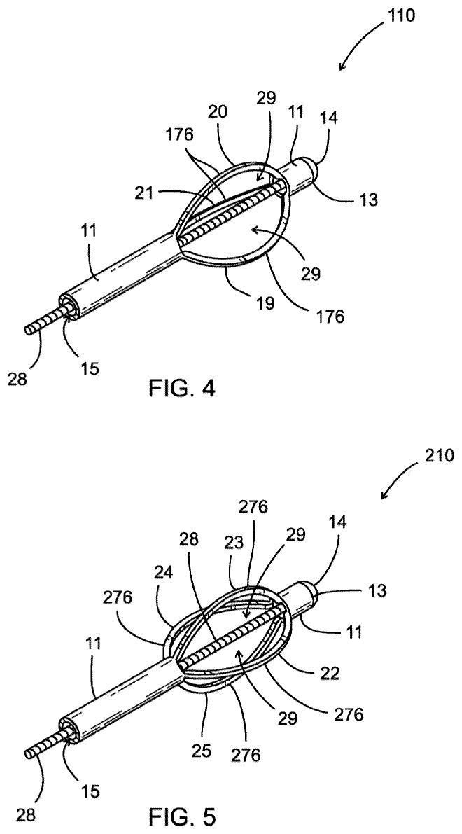

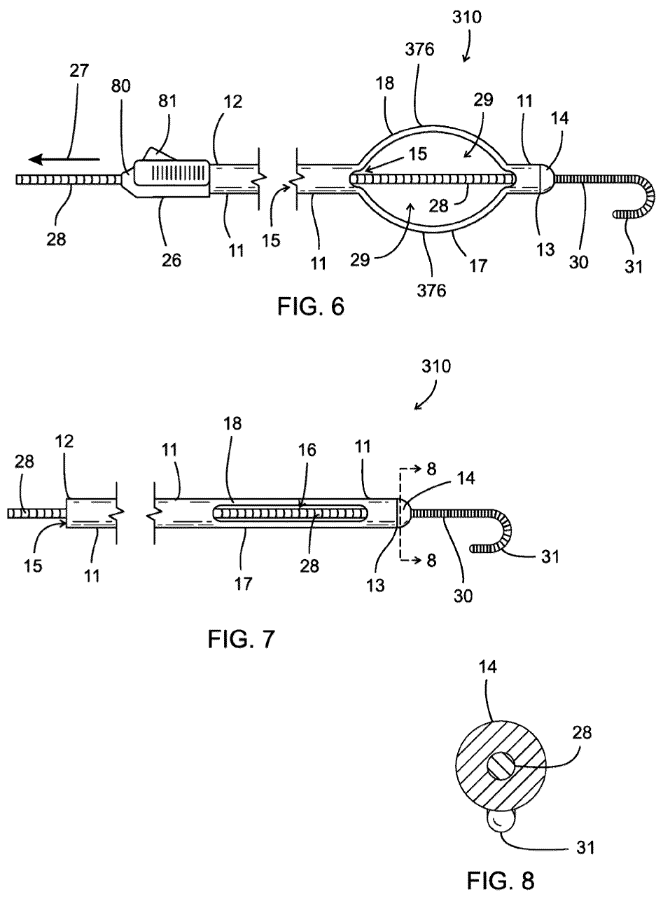

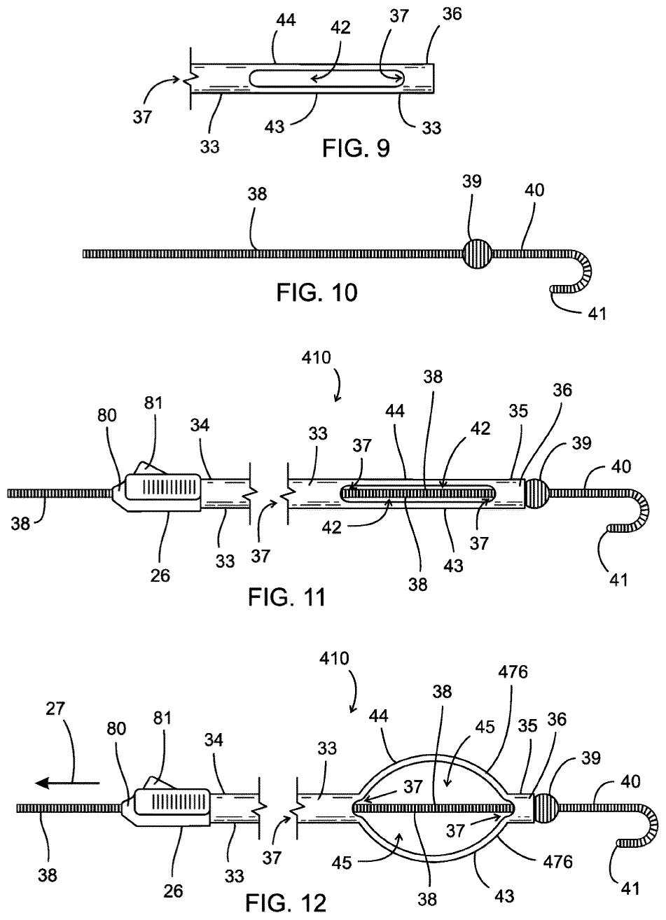

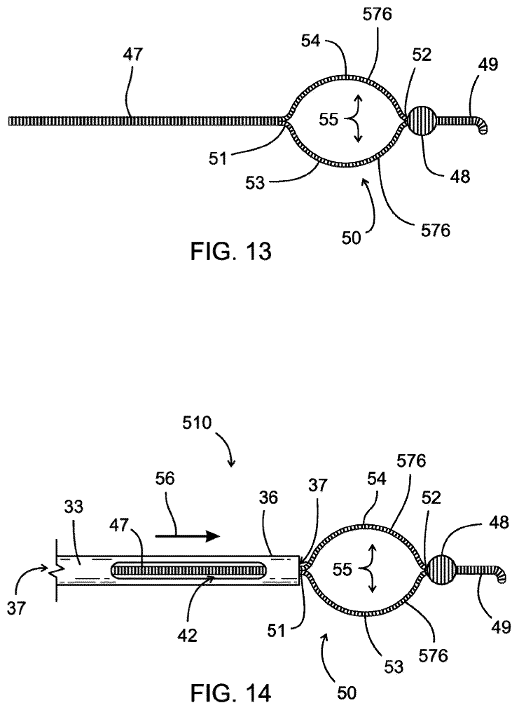

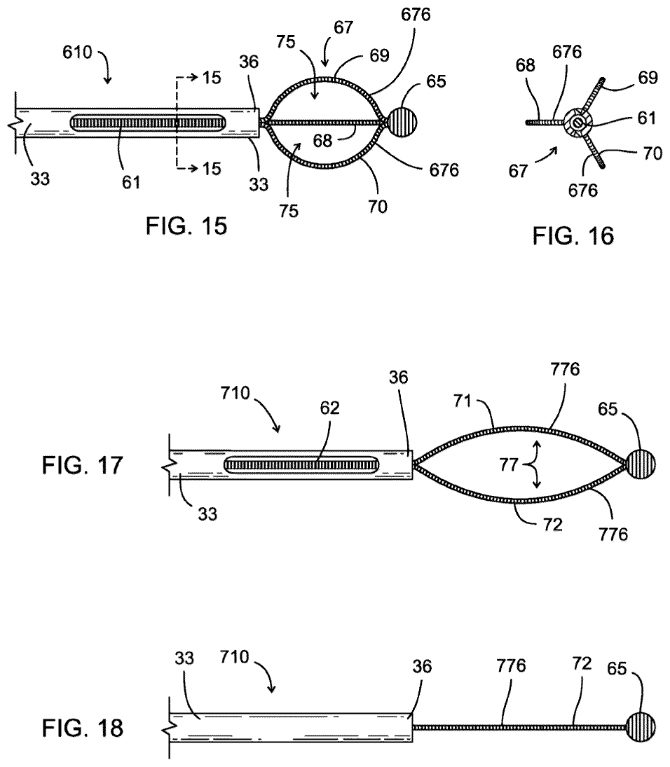

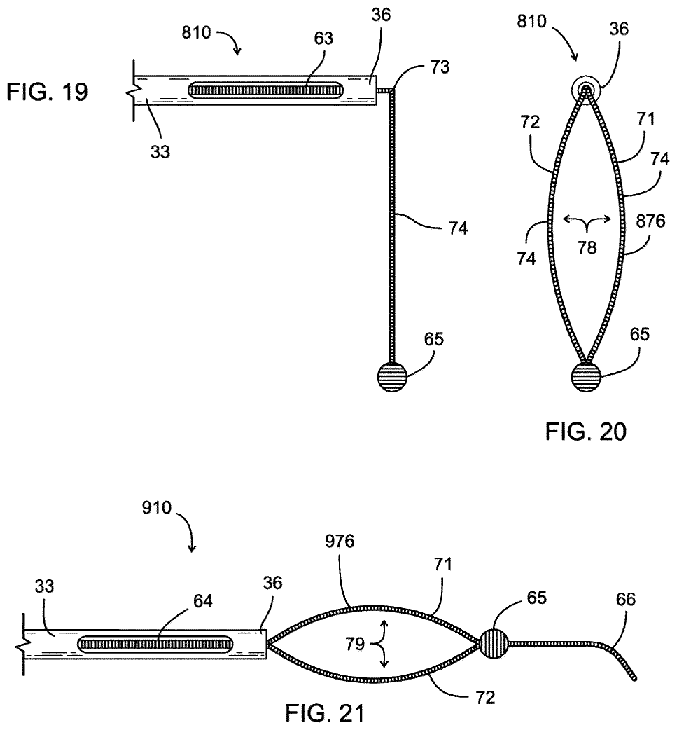

FIGS. 1-3 show a first embodiment of the apparatus of the present invention, designated generally by the numeral 10. A second embodiment is shown in FIG. 4, designated generally by the numeral 110. A third embodiment is shown in FIG. 5, designated generally by the numeral 210. A fourth embodiment is shown in FIGS. 6-8, designated generally by the numeral 310. A fifth embodiment is shown in FIGS. 9-12, designated generally by the numeral 410. A sixth embodiment is shown in FIGS. 13-14, designated generally by the numeral 510. A seventh embodiment is shown in FIGS. 15-16, designated generally by the numeral 610. An eighth embodiment is shown in FIGS. 17-18, designated generally by the numeral 710. A ninth embodiment is shown in FIGS. 19-20, designated generally by the numeral 810. A tenth embodiment is shown in FIG. 21, designated generally by the numeral 910.

In FIGS. 1-3, guidewire and snare apparatus 10 includes a sheath 11 having internal bore or lumen 15 fitted with or containing a wire or cable (or wires or cables) 28. Sheath 11 has proximal end or end portion 12 and distal end as end portion 13. In various embodiments, an atraumatic tip 14 (e.g., hemispherical, rounded or domed) is provided at distal end or end portion 13 (see FIGS. 1, 3-5).

Sheath 11 has a longitudinally extended bore or lumen 15 that is receptive of cable or wire 28 (or cables or wires). Near distal end or distal end portion 13 of sheath 11 are slotted portions 16 which can be circumferentially spaced apart (e.g., 180 degrees apart). The slotted portions 16 are in between sheath segments 17, 18. The sheath segments 17, 18 are circumferentially spaced apart, e.g., one hundred eighty degrees apart (see FIGS. 1-3). In FIGS. 1-3, wire or cable (or wires or cables) 28 attach to, engage or connect with head or tip 14. An operator 26 (for example, a commercially available guidewire operator such as the Merit® Torque Device, catalog number MAP500/MAP501, available at merit.com/cardiac-intervention/angiography/guide-wire-accessories/merit-torque-device/) can be used to pull (or apply tension to) the cable or wire (or cables or wires) 28 in the direction of arrow 27 in FIG. 3 and relative to sheath 11. Operator may comprise handle 80 and locking button/mechanism 81. An operator featuring a locking handle is known in the art, as shown in the Teleflex Micro Elite™ Snare, commercially available at teleflex.com/usa/en/product-areas/interventional/coronary-interventions/micro-elite-snare/index.html, which is incorporated herein by reference. This application of tension to cable or wire 28 (or cables or wires) generates a bend or curve in segments 17, 18 as seen in FIG. 3. Enlarged spaces 29 are thus formed at each slotted portion 16 between each segment 17, 18 and wire(s) or cable(s) 28 to form a snare 76. When the operator 26 ceases to pull on wire(s) or cable(s) 28, memory of the segments 17, 18 causes the segments 17, 18 to return to the position of FIG. 1. The sheath 11 and segments 17, 18 is preferably made of Nitinol material (though it can be made of stainless steel or other similar metal). Spaces 29 enable a snaring of an item to be retrieved (e.g., vena cava filter, foreign object, another guidewire, retrievable stent). When tension is removed from cable or wire 28, a desired feature of an item (i.e. IVC filter hook) or an item (i.e. guidewire, foreign body retrieval such as catheter fragment) is snared by segments 17, 18 of sheath 11. The item is then pulled into a larger separate sheath/retrieval sheath/retrieval catheter, which can be, for example, located close or some distance from the captured object. Alternatively, or at the same time, the retrieval sheath/retrieval catheter may be advanced forward so as to capture the apparatus of the present invention. A retrieval sheath can be used such as is known in the art. For example, see retrieval sheath shown in Bard® Snare Retrieval Kit (PF10009-BAW1313100R2 Bard Snare Retrieval Kit). FIG. 2 is a schematic, sectional view taken along lines 2-2 of FIG. 1.

A second embodiment is shown in FIG. 4 and a third embodiment is shown in FIG. 5. The embodiments of FIGS. 4 and 5 are the same as the first embodiment shown in FIGS. 1-3 except that the number of sheath segments and thus the number of slotted portions and the number of enlarged spaces have changed. For example, rather than have two sheath segments 17, 18 as seen in the first embodiment shown in FIGS. 1 and 3 (and thus two slotted portions 16), the embodiment shown in FIG. 4, designated generally by the numeral 110, shows that there can be three sheath segments 19, 20, 21 (and thus three slotted portions 16). FIG. 4, like FIG. 3, shows the apparatus with tension to cable or wire 28 applied so as to form enlarged spaces 29 at each slotted portion 16 between each segment 19, 20, 21 and wire(s) or cable(s) 28 to form snare 176. In FIG. 4, only two enlarged spaces 29 are labeled, though three are shown). Another embodiment is shown in FIG. 5, depicted generally by the numeral 210. FIG. 5 shows that there can be four sheath segments 22, 23, 24, 25 (and thus four slotted portions 16). In FIG. 5, the four slotted segments form four spaced apart sections 29 between segments 22-25 and wire(s) or cable(s) 28 when tension to cable or wire 28 is applied, in the same manner as in FIGS. 3 and 4. In FIG. 5, only two enlarged spaces 29 are labeled, though four are shown). Segments 19, 20, 21 can be spaced apart circumferentially (e.g., 120 degrees apart) as shown in FIG. 4. Segments 22, 23, 24, 25 can be circumferentially apart (e.g., 90 degrees apart) as shown in FIG. 5. Segments 19, 20, and 21 of FIG. 4 form snare 176 in the same manner that segments 17, 18 form snare 76 in the first embodiment. Similarly, segments 22, 23, 24 and 25 form snare 276 in the third embodiment shown in FIG. 5.

A fourth embodiment is shown in FIGS. 6-8, designated generally by the numeral 310. Guidewire and snare apparatus 310 is similar to the embodiment of FIGS. 1-5 but adds an optional extension 30 to head or tip 14. Although extension 30 is shown added to the first embodiment shown in FIGS. 1-3, it could likewise be added to the second and third embodiments of FIGS. 4 and 5. Extension 30 can be provided with a J-tip or bent portion 31 or otherwise angled tip. Extension 30 is rigidly affixed to head or tip 14 so that when tension is applied to wire or cable 28 in direction 27, extension 30 is not affected. Extension 30 can be an extension of wire or cable 28 or it can be a separate wire or cable. In either case, it is rigidly affixed to head 14. As shown by FIGS. 6 and 7, the length and position of extension 30 is unaffected by the adding of tension to wire or cable 28. FIG. 8 is a sectional view taken along lines 8-8 of FIG. 7. Extension 30 and J-tip or bent portion 31 provide the guidewire and snare apparatus 310 with a soft/flexible tip as can be found in guidewire tips in the art. For example, see J-tip guidewires, which provide a soft tip and a way to navigate through vasculature, also in combination with a guiding sheath. For examples of J-tip guidewires, see J-tip guidewires offered by BD at bd.com/en-us/offerings/capabilities/urology-and-kidney-health/kidney-and-stone-management/urological-guidewires/j-tip-guidewires. In the fourth embodiment shown in FIG. 6, snare 376 is formed in the same manner as snares 76, 176 and 276 of FIGS. 3-5.

In FIGS. 9-12, a fifth embodiment is designated generally by the numeral 410. Guidewire and snare apparatus or convertible guidewire apparatus 410 provides sheath 33 having proximal end or end portion 34 and distal end or end portion 35. A head 36 is provided at distal end or end portion 35. In FIGS. 9-12, head 36 can be a part of sheath 33. Sheath 33 has an open-ended bore 37 that enables wire(s) or cable(s) 38 to extend from operator 26 to and through sheath 33 and beyond distal end portion 35 and head 36 as shown in FIG. 11. Cable(s) or wire(s) 38 have stop 39 which can be a spherical shape. Sheath 33 has slotted portions 42 as with the embodiment of FIGS. 1-8. Cable(s) or wire(s) 38 can have extension 40 with J-tip, bend or bent portion 41.

When cable 38 is pulled by operator 26 in the direction of arrow 27 while sheath 33 is held or maintained in a stationary position, stop 39 engages head 36 to apply compression to sheath 33. This action expands or bends sheath segments 43, 44 to the curved position of FIG. 12, forming snare 476. Enlarged spaces 45 are thus formed for snaring a desired item in between each curved segment 43, 44 in FIG. 12 and wire or cable 38. As with the embodiments of FIGS. 1-8, when tension is removed from cable or wire 38, the sheath segments 43, 44 have memory which returns segments 43, 44 to the position of FIG. 11 which snares a desired object in between segments 43, 44 and cable 38. Once a physician or technician believes they captured the desired item, they can pull on the snare 476 gently to see if the item follows. If the item follows, the physician or technician knows the item is properly snared. Additionally, x-ray imaging can be used, for example at multiple angles, to confirm the desired item is properly snared.

FIGS. 13-14 show a sixth embodiment of the apparatus of the present invention, designated generally by the numeral 510. In FIGS. 13-14, convertible guidewire apparatus 510 has a sheath 33 similar to the sheath 33 of FIGS. 9, 11-12. If the wire 47 is pulled proximally, a snare (e.g., snare 476 of FIG. 12) can be formed from the sheath 33 as in the embodiments shown in FIGS. 1-12. In the sixth embodiment, wire 47 has stop 48 and extension 49. Wire 47 has a bifurcation 50 that begins at 51 and ends at 52 next to stop 48 as seen in FIGS. 13-14. The bifurcation 50 provides wire segments 53, 54 with enlarged space 55 in between wire segments 53, 54. The embodiment of FIGS. 13-14 enables a physician or technician to snare a desired object with wire segments 53, 54 at a position distally of head 36. Wire segments 53, 54 would have memory which can be achieved with or without shape-memory materials (e.g. nitinol, stainless steel, etc.) so that when cable 47 is advanced in the direction of arrows 56 in FIG. 14 to the position shown in FIG. 14, the wire segments expand to the curved or bent position shown in FIGS. 13 and 14, forming snare 576. A selected object would be snared by wire segments 53, 54 as the wire 47 is pulled into sheath 33 which collapses wire segments 53, 54.

FIGS. 15-16 show a seventh embodiment, designated by the numeral 610. FIGS. 17 and 18 show an eighth embodiment, designated by the numeral 710. FIGS. 19 and 20 show a ninth embodiment, designated generally by the numeral 810. FIG. 21 shows a tenth embodiment, designated generally by the numeral 910. The embodiments of FIGS. 15-21 are similar to the embodiment of FIGS. 13-14 in that sheath 33 having head 36 is employed. In FIGS. 15-21, wires 61, 62, 63, 64 each have a stop 65. Wire 61 has a trifurcated end portion 67 having curved sections 68-70 (see FIGS. 15-16) which form snare 676. An enlarged space 75 is formed between the curved sections 68-70 for enabling the sections 68, 69, 70 to snare a selected object. FIGS. 17-18 show wire 62 with longer curved sections 71, 72 that have less curvature but are longer than the curved sections 68, 69, and 70 of FIG. 14. As seen in FIG. 17, sections 71, 72 and enlarged space 77 form snare 776.

In FIGS. 19-20, the wire 63 has a bend at 73. The usefulness of bent or straight wire depends on the anatomy, the object being retrieved, and how the object is positioned/angled relative to the anatomy and the snare; having different versions of the device allows a physician to select the best approach based on the aforementioned factors. The section 74 of wire distally of bend 73 can be a bifurcation or trifurcation (e.g., FIGS. 15, 16, 17, 18) and is shown as a bifurcation in FIG. 20. As shown in FIG. 20, the section 74 of wire distally of bend 73 can form a snare 876 comprising curved wire segments 71, 72. An enlarged space 78 is formed between the curved segments 71, 72 for enabling the segments 71, 72 of snare 876 to snare a selected object. In FIG. 21, wire 64 has extension 66 distally of stop 65 which is distally of snare 976 comprising curved wire segments 71, 72 and enlarged space 79. Extension 66 can be straight or have a bend or J-tip portion as seen in FIG. 21. The embodiments shown in FIGS. 13-21 are thus able to form two different snares, one snare comprising sheath members separated by slotted portions (e.g., as with FIGS. 1-12) if the wire 61, 62, 62, 64 is pulled proximally and one comprising the inner wire which forks, if the wire is pushed distally from the sheath (e.g., FIGS. 15-21). These embodiments thus allow for snares of different sizes and different shapes to be formed using the same apparatus, depending on the particular needs at hand.

Without limitation, the guidewire and snare apparatus/convertible snare apparatus can be characterized by one or more of the following statements.

Statement 1: A surgical guidewire and snare apparatus, comprising:
a) an elongated sheath having a central, longitudinal bore and proximal and distal sheath ends.
b) a head at the sheath distal end;
c) a wire or cable that occupies said central, longitudinal bore, said wire or cable having a proximal wire or cable and a distal wire or cable end;
d) the wire or cable distal end having an engagement with the head;
e) slotted portions on the sheath at the sheath distal end;
f) spaced apart sections of the sheath next to the slotted portions; and
g) wherein applying tension to the wire or cable proximal end pulls the head proximally to enlarge the slotted portions and curve the spaced apart sheath sections next to the slotted portions.

Statement 2: The surgical guidewire and snare apparatus of the preceding statement wherein the wire or cable distal end is attached to the sheath distal end.

Statement 3: The surgical guidewire and snare apparatus of any preceding statement wherein there are two sheath sections next to the slotted portions.

Statement 4: The surgical guidewire and snare apparatus of statements 1 to 3 wherein there are three sheath sections next to the slotted portions.

Statement 5: The surgical guidewire and snare apparatus of statements 1 to 3 wherein there are four sheath sections next to the slotted portions.

Statement 6: The surgical guidewire and snare apparatus of any preceding statement wherein the sheath head has a longitudinal bore and the wire or cable extends through the sheath head bore, wherein the wire or cable has an enlarged stop member positioned distally of the head that is too large to pass through the said sheath head bore.

Statement 7: The surgical guidewire and snare apparatus of any preceding statement wherein the wire or cable extends through the head and further comprising a curved j-tip portion on the wire or cable distally of the head.

Statement 8: The surgical guidewire and snare apparatus of statement 6 wherein the wire or cable extends through the head and further comprising a curved j-tip portion on the wire or cable distally of the stop member.

Statement 9: The surgical guidewire and snare apparatus of any preceding statement wherein the wire or cable has two or more separable sections at the wire or cable distal end portion.

Statement 10: The surgical guidewire and snare apparatus of any preceding statement wherein the sheath head has a longitudinal bore and the wire or cable extends through the sheath head bore, wherein the wire or cable has an enlarged stop member positioned distally of the head that is too large to pass through said sheath head bore.

Statement 11: A surgical guidewire and snare apparatus, comprising:
a) an elongated sheath having a central, longitudinal bore and proximal and distal sheath ends;
b) a head at the sheath distal end;
c) a wire or cable that occupies said central, longitudinal bore, said wire or cable having a proximal wire or cable end and a distal wire or cable end;
d) wherein the wire or cable extends through the head and distally beyond the head;
e) slotted portions on the sheath at the sheath distal end;
f) spaced apart sections of the sheath next to the slotted portions; and
g) wherein applying tension to the wire or cable proximal end pulls the head proximally to enlarge the slotted portions and curve the spaced apart sheath sections next to the slotted portions.

Statement 12. The surgical guidewire and snare apparatus of statement 11 wherein there are multiple said wires or cables in a sheath bore.

Statement 13. The surgical guidewire and snare apparatus of one or more statements 11-12 wherein said wire or cable bifurcates into two wire or cable segments when the wire or cable is extended distally of the sheath head.

Statement 14: The surgical guidewire and snare apparatus of one or more statements 11-13 wherein there are two sheath sections next to the slotted portions.

Statement 15: The surgical guidewire and snare apparatus of one or more statements 11-13 wherein there are three sheath sections next to the slotted portions.

Statement 16: The surgical guidewire and snare apparatus of one or more statements 11-13 wherein there are four sheath sections next to the slotted portions.

Statement 17: The surgical guidewire and snare apparatus of one or more statements 11-16 wherein the sheath sections are circumferentially spaced apart.

Statement 18: The surgical guidewire and snare apparatus of one or more statements 11-17 wherein the sheath head has a longitudinal bore and the wire or cable extends through the sheath head bore, wherein the wire or cable has an enlarged stop member positioned distally of the head that is too large to pass through the said sheath head bore.

Statement 19: A surgical guidewire and snare apparatus, comprising:
a) an elongated sheath having a central, longitudinal bore and proximal and distal sheath ends;
b) a head at the sheath distal end;
c) a wire or cable that occupies said central, longitudinal bore, said wire or cable having a proximal wire or cable end and a distal wire or cable end;
d) wherein the wire or cable extends through the head and distally beyond the head;
e) slotted portions on the sheath at the sheath distal end;
f) spaced apart sections of the sheath next to the slotted portions;
g) wherein applying tension to the wire or cable proximal end pulls the head proximally to enlarge the slotted portions and curve the spaced apart sheath sections next to the slotted portions; and
h) said distal wire or cable end being bifurcated to form a wire or cable snare when said distal wire or cable end is advanced distally of said head.

Statement 20: A surgical guidewire and snare apparatus, comprising:
a) an elongated sheath having a central, longitudinal bore and proximal and distal sheath ends;
b) a head at the sheath distal end;
c) a wire or cable that occupies said central, longitudinal bore, said wire or cable having a proximal wire or cable end and a distal wire or cable end;
d) wherein the wire or cable extends through the head and distally beyond the head; e) slotted portions on the sheath at the sheath distal end;
f) spaced apart sections of the sheath next to the slotted portions;
g) wherein applying tension to the wire or cable proximal end pulls the head proximally to enlarge the slotted portions and curve the spaced apart sheath sections next to the slotted portions; and
h) wherein said distal wire or cable end forms a wire or cable snare when said distal wire or cable end is advanced distally of said head.

Statement 21: The apparatus of statement 20 wherein said distal wire or cable trifurcates into three wires or cables.

Statement 22. The apparatus of statement 21 wherein the three wires or cables comprising the wire or cable snare are approximately 120 degrees apart from each other when the wire or cable snare is formed by advancing the distal wire or cable distally of the head.

Statement 23: The apparatus of one or more statements 19-22 wherein the sheath head has a longitudinal bore and the distal wire or cable extends through the sheath head bore, wherein the distal wire or cable has an enlarged stop member positioned distally of the head that is too large to pass through said sheath head bore.

Statement 24: The apparatus of statement 23 further comprising a wire or cable extending distally of the stop member.

Statement 25: The apparatus of statement 24 wherein said wire or cable extending distally of the stop member is a straight.

Statement 26: The apparatus of statement 24 wherein said wire or cable extending distally of the stop member has a j-tip portion.

The following is a list of parts and materials suitable for use in the present invention:

PARTS LIST:

| PART | |
|---|---|
| NUMBER | DESCRIPTION |
| 10 | guidewire and snare apparatus/convertible |
| | snare apparatus |
| 11 | sheath |
| 12 | proximal end/proximal end portion |
| 13 | distal end/distal end portion |
| 14 | hemispherical tip/rounded tip/head |
| 15 | sheath longitudinal bore/lumen |
| 16 | slotted portion |
| 17 | sheath segment |
| 18 | sheath segment |
| 19 | sheath segment |
| 20 | sheath segment |
| 21 | sheath segment |
| 22 | sheath segment |
| 23 | sheath segment |
| 24 | sheath segment |
| 25 | sheath segment |
| 26 | operator |
| 27 | arrow |
| 28 | wire/cable/wires/cables |
| 29 | enlarged space/space |
| 30 | extension |
| 31 | hook/bend/bent portion |
| 33 | sheath |
| 34 | proximal end |
| 35 | distal end |
| 36 | head |
| 37 | open ended bore |
| 38 | wire/cable |
| 39 | stop member |
| 40 | extension |
| 41 | hook/bend |
| 42 | slotted portion |
| 43 | sheath segment |
| 44 | sheath segment |
| 45 | enlarged space |
| 47 | wire/cable |
| 48 | stop member |
| 49 | extension |
| 50 | bifurcation |
| 51 | position |
| 52 | position |
| 53 | wire segment |
| 54 | wire segment |
| 55 | enlarged space |
| 56 | arrow |
| 61 | wire |
| 62 | wire |
| 63 | wire |
| 64 | wire |
| 65 | stop |
| 66 | extension |
| 67 | end portion |
| 68 | curved wire segment |
| 69 | curved wire segment |
| 70 | curved wire segment |
| 71 | curved wire segment |
| 72 | curved wire segment |
| 73 | bend |
| 74 | section |
| 75 | snare space/space |
| 76 | snare |
| 77 | enlarged space |
| 78 | enlarged space |
| 79 | enlarged space |
| 80 | handle |
| 81 | locking button |
| 110 | guidewire and snare apparatus/convertible |
| | guidewire apparatus |
| 176 | snare |
| 210 | guidewire and snare apparatus/convertible |
| | guidewire apparatus |
| 276 | snare |
| 310 | guidewire and snare apparatus/convertible |
| | guidewire apparatus |
| 376 | snare |
| 410 | guidewire and snare apparatus/convertible |
| | guidewire apparatus |
| 476 | snare |
| 510 | guidewire and snare apparatus/convertible |
| | guidewire apparatus |
| 576 | snare |
| 610 | guidewire and snare apparatus/convertible |
| | guidewire apparatus |
| 676 | snare |
| 710 | guidewire and snare apparatus/convertible |
| | guidewire apparatus |
| 776 | snare |
| 810 | guidewire and snare apparatus/convertible |
| | guidewire apparatus |
| 876 | snare |
| 910 | guidewire and snare apparatus/convertible |
| | guidewire apparatus |

The foregoing embodiments are presented by way of example only; the scope of the present invention is to be limited only by the following claims.

1. A surgical guidewire and snare apparatus, comprising:
a) an elongated sheath having a central, longitudinal bore and proximal and distal sheath ends;
b) a head at the sheath distal end;
c) a wire or cable that occupies said central, longitudinal bore, said wire or cable having a proximal wire or cable and a distal wire or cable end;
d) the wire or cable distal end having an engagement with the head;
e) slotted portions on the sheath at the sheath distal end;
f) spaced apart sections of the sheath next to the slotted portions; and
g) wherein applying tension to the wire or cable proximal end pulls the head proximally to enlarge the slotted portions and curve the spaced apart sheath sections next to the slotted portions.

2. The surgical guidewire and snare apparatus of claim 1 wherein the wire or cable distal end is attached to the sheath distal end.

3. The surgical guidewire and snare apparatus of claim 1 wherein there are two sheath sections next to the slotted portions.

4. The surgical guidewire and snare apparatus of claim 1 wherein there are three sheath sections next to the slotted portions.

5. The surgical guidewire and snare apparatus of claim 1 wherein there are four sheath sections next to the slotted portions.

6. The surgical guidewire and snare apparatus of claim 1 wherein the sheath head has a longitudinal bore and the wire or cable extends through the sheath head bore, wherein the wire or cable has an enlarged stop member positioned distally of the head that is too large to pass through the said sheath head bore.

7. The surgical guidewire and snare apparatus of claim 1 wherein the wire or cable extends through the head and further comprising a curved j-tip portion on the wire or cable distally of the head.

8. The surgical guidewire and snare apparatus of claim 6 wherein the wire or cable extends through the head and further comprising a curved j-tip portion on the wire or cable distally of the stop member.

9. The surgical guidewire and snare apparatus of claim 1 wherein the wire or cable has two or more separable sections at the wire or cable distal end portion.

10. The surgical guidewire and snare apparatus of claim 9 wherein the sheath head has a longitudinal bore and the wire or cable extends through the sheath head bore, wherein the wire or cable has an enlarged stop member positioned distally of the head that is too large to pass through said sheath head bore.

11. A surgical guidewire and snare apparatus, comprising:
a) an elongated sheath having a central, longitudinal bore and proximal and distal sheath ends;
b) a head at the sheath distal end;
c) a wire or cable that occupies said central, longitudinal bore, said wire or cable having a proximal wire or cable end and a distal wire or cable end;
d) wherein the wire or cable extends through the head and distally beyond the head;
e) slotted portions on the sheath at the sheath distal end;
f) spaced apart sections of the sheath next to the slotted portions; and
g) wherein applying tension to the wire or cable proximal end pulls the head proximally to enlarge the slotted portions and curve the spaced apart sheath sections next to the slotted portions.

12. The surgical guidewire and snare apparatus of claim 11 wherein there are multiple said wires or cables in a sheath bore.

13. The surgical guidewire and snare apparatus of claim 11 wherein said wire or cable bifurcates into two wire or cable segments when the wire or cable is extended distally of the sheath head.

14. The surgical guidewire and snare apparatus of claim 11 wherein there are two sheath sections next to the slotted portions.

15. The surgical guidewire and snare apparatus of claim 11 wherein there are three sheath sections next to the slotted portions.

16. The surgical guidewire and snare apparatus of claim 11 wherein there are four sheath sections next to the slotted portions.

17. The surgical guidewire and snare apparatus of claim 11 wherein the sheath sections are circumferentially spaced apart.

18. The surgical guidewire and snare apparatus of claim 11 wherein the sheath head has a longitudinal bore and the wire or cable extends through the sheath head bore, wherein the wire or cable has an enlarged stop member positioned distally of the head that is too large to pass through the said sheath head bore.

19. A surgical guidewire and snare apparatus, comprising:
a) an elongated sheath having a central, longitudinal bore and proximal and distal sheath ends;
b) a head at the sheath distal end;
c) a wire or cable that occupies said central, longitudinal bore, said wire or cable having a proximal wire or cable end and a distal wire or cable end;
d) wherein the wire or cable extends through the head and distally beyond the head;
e) slotted portions on the sheath at the sheath distal end;
f) spaced apart sections of the sheath next to the slotted portions;
g) wherein applying tension to the wire or cable proximal end pulls the head proximally to enlarge the slotted portions and curve the spaced apart sheath sections next to the slotted portions; and
h) said distal wire or cable end being bifurcated to form a wire or cable snare when said distal wire or cable end is advanced distally of said head.

20. A surgical guidewire and snare apparatus, comprising:
a) an elongated sheath having a central, longitudinal bore and proximal and distal sheath ends;
b) a head at the sheath distal end;
c) a wire or cable that occupies said central, longitudinal bore, said wire or cable having a proximal wire or cable end and a distal wire or cable end;
d) wherein the wire or cable extends through the head and distally beyond the head;
e) slotted portions on the sheath at the sheath distal end;
f) spaced apart sections of the sheath next to the slotted portions;
g) wherein applying tension to the wire or cable proximal end pulls the head proximally to enlarge the slotted portions and curve the spaced apart sheath sections next to the slotted portions; and
h) wherein said distal wire or cable end forms a wire or cable snare when said distal wire or cable end is advanced distally of said head.

21. The apparatus of claim 20 wherein said distal wire or cable trifurcates into three wires or cables.

22. The apparatus of claim 21 wherein the three wires or cables comprising the wire or cable snare are approximately 120 degrees apart from each other when the wire or cable snare is formed by advancing the distal wire or cable distally of the head.

23. The apparatus of claim 22 wherein the sheath head has a longitudinal bore and the distal wire or cable extends through the sheath head bore, wherein the distal wire or cable has an enlarged stop member positioned distally of the head that is too large to pass through said sheath head bore.

24. The apparatus of claim 23 further comprising a wire or cable extending distally of the stop member.

25. The apparatus of claim 24 wherein said wire or cable extending distally of the stop member is a straight.

26. The apparatus of claim 24 wherein said wire or cable extending distally of the stop member has a j-tip portion.

27. The inventions substantially as shown and/or described herein.
